(19) United States
(12) Patent Application Publication
(10) Pub. No.: US 2004/0092992 A1
(43) Pub. Date: May 13, 2004

(54) Disposable battery powered rotary tissue cutting instruments and methods therefor

(76) Inventors: Kenneth Adams, Jacksonville, FL (US); Earnest Ferrell, Orange Park, FL (US); Kevin McFarlin, Jacksonville, FL (US)

Correspondence Address: EPSTEIN & GERKEN, Suite 340, 1901 Research Boulevard, Rockville, MD, 20850-3164, US

(21) Appl. No.: 10/278,012

(22) Filed: Oct. 23, 2002

(51) Int. Cl. A61B 17/32

(52) U.S. Cl. 606/180

(57) ABSTRACT

A disposable battery powered rotary tissue cutting instrument for soft tissue removal includes a blade attached to a powered surgical handpiece. The blade comprises an elongate tubular outer member and an inner member rotatably disposed within the outer member to cut anatomical tissue. The handpiece contains a motor having a drive shaft coupled in driving engagement with the inner member and a battery unit for supplying electric current to rotate the drive shaft to effect rotation of the inner member within the outer member. The inner member is continuously rotated in one direction or is continuously oscillatorily rotated in forward and reverse directions. The instrument is disposable after single patient use and may include a use-limiting unit for automatically disabling the instrument to prevent its reuse after completion of a surgical procedure performed using the instrument on a single patient. The blade may be used for electric cautery.

### BACKGROUND OF THE INVENTION

1. Field of the Invention

The present invention relates generally to battery powered rotary tissue cutting instruments for surgical use and, more particularly, to disposable battery powered rotary tissue cutting instruments for removing soft tissue in a surgical procedure performed on a single patient, especially surgical procedures of the ears, nose and throat, and to methods of surgery using a disposable battery-powered rotary tissue cutting instrument.

2. Brief Discussion of the Related Art

Rotary tissue cutting instruments comprising an elongate outer tubular member and an elongate inner member rotatably disposed in the outer member to form a blade for cutting anatomical tissue have become well accepted for use in various surgical procedures. Typically, the inner member has a distal end with a cutting edge and the outer member has an opening through which the cutting edge is exposed to access anatomical tissue to be cut by the cutting edge when the inner member is rotated within the outer member. In some instances, the distal end of the outer member has a cutting edge cooperable with the cutting edge of the inner member to cut the anatomical tissue as the inner member is rotated. The outer and inner members ordinarily have proximal ends adapted for releasable coupling with a powered surgical handpiece used to rotate the inner member relative to and within the outer member. Many rotary tissue cutting instruments provide for aspiration of anatomical debris through the instrument and/or irrigation at the operative site via an irrigating or flushing fluid supplied along the instrument.

Rotary tissue cutting instruments designed for soft tissue removal have advantageously been used to remove soft tissue in surgical procedures of the ears, nose and/or throat, and especially in tonsillectomy, adenoidectomy and sinus procedures. Rotary tissue cutting instruments that are advantageous for removing soft tissue in procedures of the ears, nose and/or throat (ENT) are represented by U.S. Pat. No. 5,922,003 to Anctil et al and by the RADenoid® Blade, the RAD® 40 Curved Blade and the RAD 60 X-TREME™ Curved Blade of Medtronic Xomed Surgical Products, Inc. The blade disclosed in the Anctil et al patent is particularly advantageous for soft tissue removal in adenoidectomy as well as tonsillectomy procedures, while the RAD® 40 Curved Blade and the RAD 60 X-TREME™ Curved Blade are particularly advantageous for soft tissue removal in sinus procedures.

The blades of rotary tissue cutting instruments used to remove soft tissue in ENT procedures may be longitudinally or axially straight or may be longitudinally or axially bent, angled or curved depending on the location of the anatomical tissue to be accessed. The blades are typically characterized by a side-facing opening at the distal end of the inner member, a cutting edge extending along a peripheral edge of the opening, a side-facing opening at the distal end of the outer member adjacent the inner member opening, and a cutting edge extending along a peripheral edge of the outer member opening. The cutting edges of the inner and outer members typically comprise a plurality of cutting teeth extending along the peripheral edges of the openings, respectively. The inner member cutting edge moves past the outer member cutting edge to continuously remove, cut or shave anatomical tissue when the inner member is rotated relative to and within the outer member. Rotary tissue cutting instruments of the latter type allow precise progressive removal of soft anatomical tissue while simultaneous suction or aspiration evacuates blood and tissue from the operative site for enhanced visualization. Well-defined portions of anatomical tissue can be precisely shaved away exactly where the blade is placed, resulting in increased surgical accuracy. This is in contrast to the cutting action obtained with rotary surgical drills conventionally used to cut hard anatomical tissue such as bone and with dental drills used to cut dentin. Rotary tissue cutting instruments for soft tissue removal, as represented by the Anctil et al patent, the RADenoid® Blade, the RAD® 40 Curved Blade and the RAD 60 X-TREME™ Curved Blade, typically permit irrigating or flushing fluid to be supplied along the instrument to the operative site. Irrigating fluid can be supplied along the instrument in various ways including through the inner member, between the inner member and the outer member, and externally alongside the outer member.

The powered surgical handpieces of rotary tissue cutting instruments ordinarily contain a motor for driving or rotating the inner member relative to and within the outer member. The motor is usually powered via a remote console to which the motor is electrically connected by a heavy power cord or cable extending from the handpiece to the console as illustrated by U.S. Pat. No. 5,871,493 to Sjostrom et al and U.S. Pat. No. 5,632,759 to Rexroth. Operation of the motor is typically controlled by a foot switch which is also connected with the console. Powered surgical handpieces that rely on connection to a console and foot switch operation have the drawback of requiring a significant investment in expensive capital equipment in operating rooms and other surgical sites. In addition, the power cord may interfere with movements of the surgeon's hand used to manipulate the handpiece, and the presence of the power cord in the operating field may be a nuisance to the surgeon. The power cord may thusly limit the surgeon's ability to manipulate the instrument in a controlled manner, and the resulting inability to control the instrument with great precision may render surgical procedures, and especially delicate surgical procedures, more difficult. The foot pedal further complicates procedural use in that operation of the instrument must be effected and controlled remote from the handpiece, thereby requiring great coordination between hand and foot to ensure against potential surgical error.

It has been proposed to power some rotary tissue cutting instruments via batteries contained in the handpieces as represented by U.S. Pat. No. 5,796,188 to Bays, U.S. Pat. No. 4,320,761 to Haddad and U.S. Pat. No. 3,173,417 to Horner. The Bays and Horner patents disclose battery powered drills for drilling hard anatomical tissue. The drill disclosed in the Bays patent includes a booster battery disposed externally of the handpiece and connected with an external plug via an electrical cable. The Haddad patent discloses an instrument used to excise hard cataractous tissue. U.S. Pat. No. 5,249,583 to Mallaby and U.S. Pat. No. 4,461,305 to Cibley relate to battery powered biopsy instruments for obtaining discrete tissue samples but which do not operate to continuously remove, cut or shave anatomical tissue. Reusable battery powered toothbrushes have been proposed, as represented by U.S. Pat. No. 6,180,693 B1 and U.S. Pat. No. 6,000,083 to Blaustein et al and U.S. Pat. No. 5,625,916 to McDougall, but do not relate to surgical instruments or procedures.

Powered surgical handpieces, including those that are battery powered, are predominantly designed to be reusable with removably attachable disposable blades. Accordingly, the components of reusable surgical handpieces must be protected from or capable of withstanding the heat and/or chemicals of repeated sterilizations to medical standards, and this adds to the cost of reusable surgical handpieces. Seals are sometimes provided in reusable battery powered surgical handpieces to isolate the batteries from the effects of heat and/or chemicals, but sealed handpieces are more expensive due to the added design and manufacturing complexity as well as the need for additional structural components. Reusable surgical handpieces have various additional disadvantages in that, even where designed to tolerate repeated sterilizations, the mechanical and/or electrical components of the handpiece may still be degraded by the sterilization processes required for repeated surgical use. Reusable surgical handpieces may thusly present high failure rates due to the effects of repeated sterilizations. In reusable battery powered surgical handpieces, the batteries may be removed from the handpieces prior to sterilization and then reinserted in the handpieces subsequent to sterilization. However, the removal and reinsertion process increases the labor requirements and costs of surgical procedures and poses the risk that the sterilized handpiece may be contaminated during battery reinsertion. A further disadvantage of reusable battery powered surgical handpieces is that the handpiece is susceptible to power loss during a surgical procedure due to battery drain from prior repeated uses. The instrument may thusly become inoperative during the surgical procedure, requiring use of a new instrument or the installation of fresh batteries in the handpiece. The occurrence of a power loss is disruptive to the surgical procedure and may place the patient at increased risk for complications by unduly extending the time required to perform the surgical procedure.

U.S. Pat. No. 5,849,023 to Mericle discloses a tissue cutting instrument having a disposable surgical handpiece which is not battery powered. The handpiece is connected with a motor disposed in a remote motor housing, the connection being established via a flex-shaft extending from the handpiece to the motor housing. Operation of the instrument is controlled via a foot switch, and the handpiece has the same drawbacks discussed above forfoot actuated handpieces that are connected with a remote console.

In light of the above, there is a need for disposable battery powered rotary tissue cutting instruments for continuous removal of soft anatomical tissue, especially soft anatomical tissue of the ears, nose and/or throat, in which the surgical handpieces and blades of the instruments are disposable as an integral unit upon completion of a surgical procedure performed on a single patient. The need further exists for disposable rotary tissue cutting instruments for soft tissue removal that are cost-effectively designed while being entirely self-powered, without the need for any additional power or “boost” from an extraneous power source. There is an additional need for disposable battery powered rotary tissue cutting instruments having rotatable inner members capable of continuous oscillatory rotation in forward and reverse directions for amplified cutting action. There is also a need for disposable tissue cutting instruments wherein a use-limiting unit prevents reuse of the instruments after completion of a surgical procedure performed on a single patient.

### SUMMARY OF THE INVENTION

Accordingly, it is a primary object of the present invention to overcome the aforementioned disadvantages of prior rotary tissue cutting instruments for soft tissue removal.

Another object of the present invention is to provide a disposable battery powered rotary tissue cutting instrument for continuously removing soft tissue in surgical procedures, particularly surgical procedures of the ears, nose and/or throat.

A further object of the present invention is to supply a sterile battery powered rotary tissue cutting instrument ready for single patient use to remove soft tissue in surgical procedures of the ears, nose and/or throat.

It is also an object of the present invention to supply a battery powered rotary tissue cutting instrument for single patient use in surgical procedures of the ears, nose and/or throat to ensure that sufficient battery power to operate the instrument will be available throughout the surgical procedure.

An additional object of the present invention is to permit both continuous rotation in one direction and continuous oscillatory rotation of an inner member of a battery powered rotary tissue cutting instrument.

Yet another object of the present invention is to prevent reuse of a disposable battery powered rotary tissue cutting instrument upon completion of a surgical procedure performed on a single patient using the battery powered rotary tissue cutting instrument.

The present invention also has as an object to enhance the safety of surgical procedures by providing instantaneous on/off control of the cutting action in a battery powered rotary tissue cutting instrument.

Moreover, it is an object of the present invention to utilize the cutting blade of a disposable battery powered rotary tissue cutting instrument for cautery following tissue resection with the blade.

Still a further object of the present invention is to perform an adenoidectomy and /or a tonsillectomy using a disposable battery powered rotary tissue cutting instrument.

Some of the advantages of the present invention are that the need for expensive capital equipment in operating rooms and other surgical sites is eliminated; preoperative set-up for surgical procedures is reduced; the presence of a power cable extending from the surgical handpieces is eliminated; foot controlled operation of rotary tissue cutting instruments is avoided; the battery powered rotary tissue cutting instruments can be operated with a single hand via controls on the surgical handpieces; the blades may be non-removably attached to the surgical handpieces thereby avoiding the additional costs associated with removably attachable blades; the battery powered rotary tissue cutting instruments can be provided with an internal or external irrigation passage for supplying irrigating fluid through or along the blades; the battery powered rotary tissue cutting instruments can be provided with an internal or external aspiration passage for aspiration or suction through or along the blades; irrigation and/or aspiration fittings can be disposed at various locations on the instruments; the battery powered rotary tissue cutting instruments can be provided in a sterile package ready for use; the surgical handpieces are easily manipulatable by a surgeon with precision and accuracy using a single hand; various types of batteries can be used in the surgical handpieces including conventional dry cell batteries of sufficient voltage to ensure completion of a surgical procedure performed on a single patient; the surgical handpieces do not have to be sterilized subsequent to a surgical procedure; the batteries do not have to be removed from and reinserted in the handpieces subsequent to a surgical procedure; surgical procedures involving removal of soft anatomical tissue from the ears, nose and/or throat are facilitated, and tonsillectomy and adenoidectomy procedures are particularly facilitated.

These and other objects, advantages and benefits are realized with the present invention as generally characterized in a disposable battery powered rotary tissue cutting instrument for soft tissue removal comprising a blade attached to a powered surgical handpiece. The blade comprises an elongate tubular outer member and an elongate tubular inner member rotatably disposed within the outer member. The outer member extends distally from the handpiece to a distal end having a side-facing opening and a cutting edge extending along a peripheral edge of the opening. The inner member extends distally from the handpiece to a distal end having a side-facing opening adjacent the opening in the outer member and a cutting edge extending along a peripheral edge of the opening in the inner member. The cutting edge of the inner member is cooperable with the cutting edge of the outer member to cut soft anatomical tissue adjacent the opening in the outer member when the inner member is rotated relative to and within the outer member. The outer member may be longitudinally or axially straight or may be longitudinally or axially bent, curved or angled. Where the outer member is bent, curved or angled, the inner member has a flexible region adjacent the bend in the outer member and conforms to the configuration of the outer member as the inner member rotates within the outer member. The powered surgical handpiece comprises a housing fixedly mounting the outer member and rotatably mounting the inner member. A motor is disposed within the housing and includes a drive shaft in driving engagement with the inner member. A battery unit within the housing supplies electric current to the motor to rotate the drive shaft, and a control unit is disposed along an external surface of the handpiece for selectively allowing electric current to flow from the battery unit to the motor to start and stop rotation of the drive shaft. The drive shaft and, therefore, the inner member, is continuously rotated in a single direction or is continuously oscillatorily rotated in forward and reverse directions for amplified cutting action. The instrument is disposable upon completion of a surgical procedure performed with the instrument on a single patient. A use-limiting unit of the instrument automatically disables the instrument to prevent its reuse after completion of a surgical procedure performed using the instrument on a single patient. The use-limiting unit automatically prevents the flow of electric current to the motor after a predetermined amount of use of the instrument. The use-limiting unit may operate to automatically disable the instrument by opening an electric circuit of the handpiece or by causing electric current to drain from the battery. The instrument may be provided with an irrigation passage for supplying irrigating fluid through or along the instrument, and may be provided with an aspiration passage for suction through or along the instrument. The blade may be electrically coupled with an electrosurgical generator for use as a cautery.

The present invention is also generally characterized in a method of performing a surgical procedure on a patient comprising the steps of grasping a surgical handpiece of a sterile rotary tissue cutting instrument having an elongate tubular outer member extending from the handpiece and an elongate inner member extending from the handpiece and being rotatably disposed within the outer member; positioning a distal end of the outer member at an operative site in the patient's body such that an opening in the distal end is positioned adjacent anatomical tissue to be removed; causing electric current to flow from a battery unit disposed within the handpiece to a motor disposed within the handpiece and having a drive shaft in driving engagement with a proximal end of the inner member; continuously rotating the drive shaft to rotate the inner member relative to and within the outer member in response to the flow of electric current to the motor; cutting the anatomical tissue adjacent the opening with a cutting edge of the inner member that is exposed from the opening as the inner member is continuously rotated within the outer member; terminating the flow of electric current from the battery unit to the motor; stopping rotation of the drive shaft to stop rotation of the inner member in response to the termination of electric current flow to the motor; withdrawing the instrument from the patient's body; and automatically disabling the instrument subsequent to completion of the surgical procedure on the patient such that the instrument cannot be reused. Anatomical tissue cut by the instrument may be removed via aspiration through or along the instrument. Irrigating fluid may be supplied to the operative site through or along the instrument. Subsequent to the step of cutting, the instrument may be used to cauterize anatomical tissue in the patient's body.

The present invention is also generally characterized in a method of performing a surgical procedure on a patient including an adenoidectomy comprising the steps of introducing a distal end of a blade of a rotary tissue cutting instrument in the patient's nasopharynx; positioning an opening in a distal end of a tubular outer member of the blade adjacent an adenoid; causing electric current to flow from a battery unit within a surgical handpiece that is attached to the blade to a motor within the handpiece; continuously rotating a drive shaft of the motor to rotate an inner member of the blade relative to and within the outer member in response to the flow of electric current to the motor; cutting anatomical tissue of the adenoid with a cutting edge of the inner member that moves past a cutting edge of the outer member as the inner member is rotated within the outer member; terminating the flow of electric current from the battery unit to the motor; stopping rotation of the drive shaft to stop rotation of the inner member in response to the termination of electric current flow to the motor; withdrawing the blade from the nasopharynx; and disposing of the instrument after completion of the surgical procedure on the patient.

The present invention is further characterized in a method of performing a surgical procedure on a patient including a tonsillectomy comprising the steps of introducing a distal end of a blade in the patient's mouth; positioning an opening in a distal end of a tubular outer member of the blade adjacent a tonsil; causing electric current to flow from a battery unit within a surgical handpiece that is attached to the blade to a motor within the handpiece; continuously rotating a drive shaft of the motor to rotate an inner member of the blade relative to and within the outer member in response to the flow of electric current to the motor; cutting anatomical tissue of the tonsil with a cutting edge of the inner member that moves past a cutting edge of the outer member as the inner member is rotated within the outer member; terminating the flow of electric current from the battery unit to the motor; stopping rotation of the drive shaft to stop rotation of the inner member in response to the termination of electric current flow to the motor; withdrawing the blade from the patient's mouth; and disposing of the instrument after completion of the surgical procedure on the patient. The tonsillectomy can be performed separately or in conjunction with the adenoidectomy. Where the adenoidectomy and tonsillectomy are performed together, the instrument is disposed of after completion of the combined tonsillectomy and adenoidectomy procedure (T&A procedure).

### BRIEF DESCRIPTION OF THE DRAWINGS

FIG. 1 is a side view, partly in section, of a disposable battery powered rotary tissue cutting instrument according to the present invention.

FIG. 2 is a top view of the disposable battery powered rotary tissue cutting instrument.

FIG. 3 is an exploded side view of a tissue cutting blade of the disposable battery powered rotary tissue cutting instrument.

FIG. 4 is a broken longitudinal sectional view of the disposable battery powered rotary tissue cutting instrument showing attachment of the cutting blade to the surgical handpiece of the disposable battery powered rotary tissue cutting instrument.

FIG. 5 is a schematic representation for oscillatory rotation in the disposable battery powered rotary tissue cutting instrument.

FIG. 6 illustrates a representative motor drive circuit for the disposable battery powered rotary tissue cutting instrument.

FIG. 7 depicts in flow chart form a use-limiting unit of the disposable battery powered rotary tissue cutting instrument.

FIG. 8 is a perspective view showing preparation of the disposable battery powered rotary tissue cutting instrument for performance of a surgical procedure.

FIG. 9 is a broken perspective view, partly in section, illustrating use of the disposable battery powered rotary tissue cutting instrument to perform an adenoidectomy.

FIG. 10 is a broken perspective view, partly in section, depicting use of the disposable battery powered rotary tissue cutting instrument to perform a tonsillectomy.

FIG. 11 is an exploded perspective view of an alternative disposable battery powered rotary tissue cutting instrument according to the present invention.

FIG. 12 is a side view of another alternative disposable battery powered rotary tissue cutting instrument of the present invention.

FIG. 13 is an end view of the powered surgical handpiece for the disposable battery powered rotary tissue cutting instrument of FIG. 12.

FIG. 14 is a side view depicting the disposable battery powered rotary tissue cutting instrument of FIG. 12 as held in the hand of a surgeon during use.

### DESCRIPTION OF THE PREFERRED EMBODIMENTS

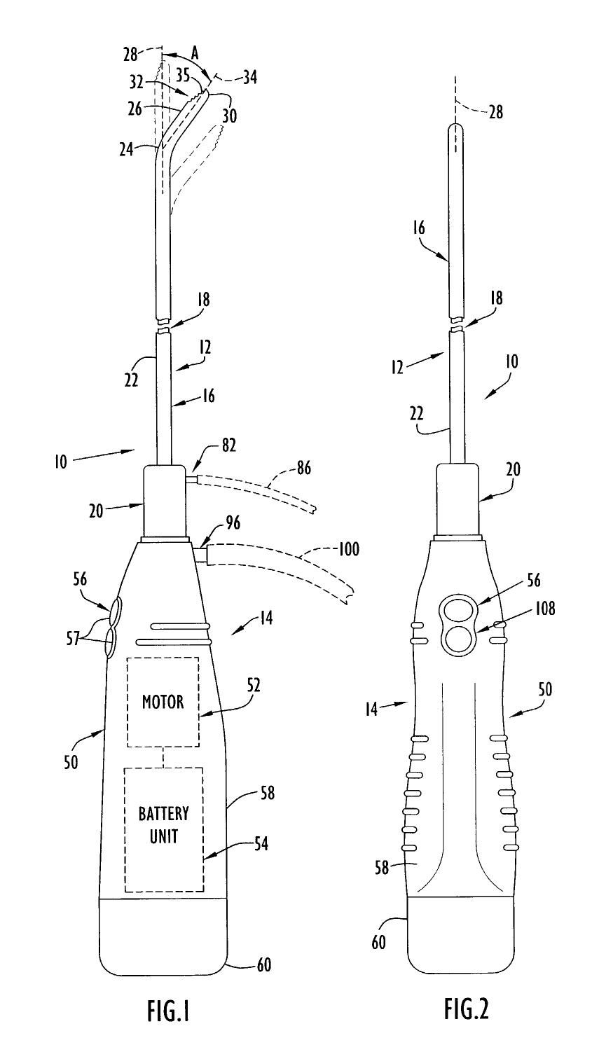

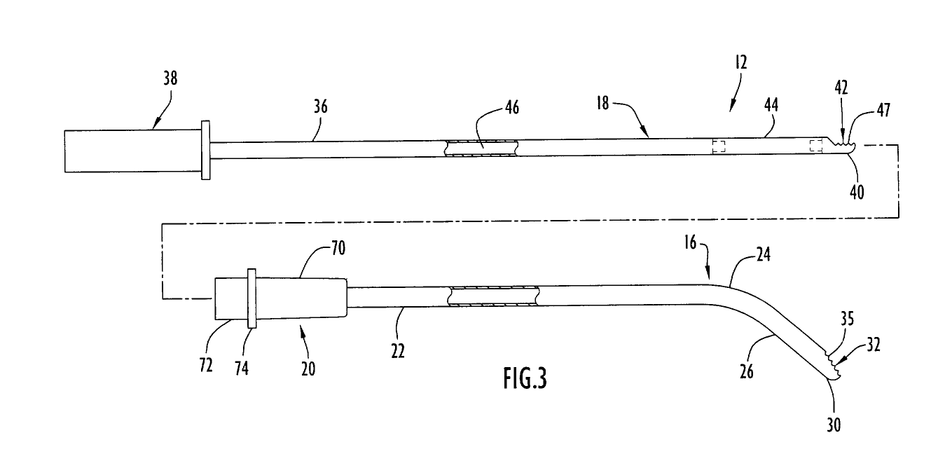

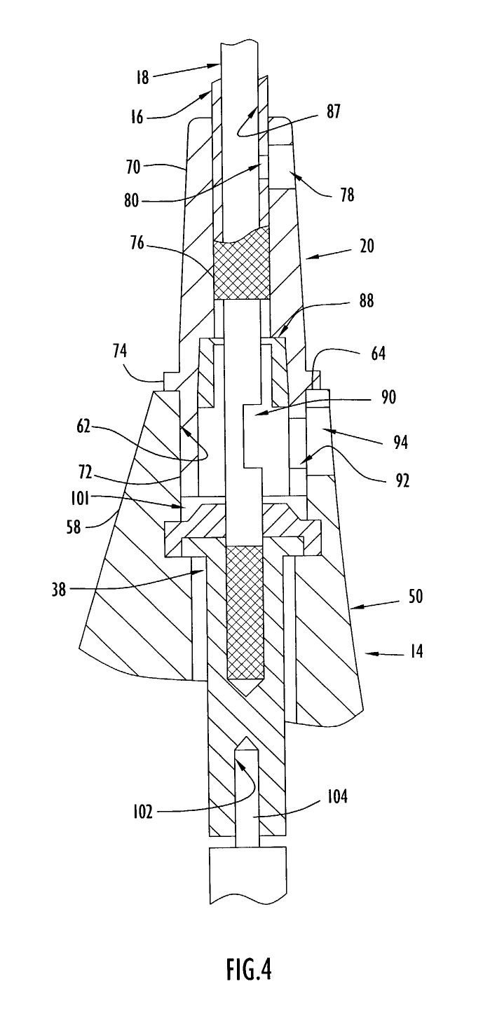

A disposable battery powered rotary tissue cutting instrument 10 according to the present invention is shown in FIGS. 1 and 2 and comprises a tissue cutting blade 12 attached to a powered surgical handpiece 14. The blade 12 is best shown in FIG. 3 and includes an elongate outer tubular member 16 and an elongate inner member 18 rotatably disposed within the outer member. Outer member 16, which may be considered an outer blade member, is an outer tubular member or sleeve having a proximal end fixedly attached to an outer member hub 20 which, in turn, is fixedly attached to the handpiece 14 as described further below. The outer member 16 can be longitudinally or axially bent, curved or angled as shown in FIGS. 1 and 3 or can be longitudinally or axially straight as shown in dotted lines in FIG. 1. The longitudinally or axially bent, curved or angled outer member 16 shown in FIGS. 1 and 3 has a proximal length portion 22 of longitudinally or axially straight configuration extending distally from the handpiece 14 to a bend, curve or angle 24 connecting the proximal length portion with a distal length portion 26 oriented at an angle A relative to the central longitudinal axis 28 of the proximal length portion. The distal length portion is of longitudinally or axially straight configuration extending distally from bend 24 to a distal end 30 having a side-facing opening 32, and the angle A is defined between a central longitudinal axis 34 of the distal length portion and the central longitudinal axis 28. The distal end 30 has a cutting edge 35 comprising a plurality of cutting teeth extending along a peripheral edge of opening 32. The outer member 16 is typically made of a medically acceptable metal such as stainless steel with the bend 24 pre-formed therein as part of the fabrication or manufacture process.

The inner member 18, as best shown in FIG. 3, may be considered an inner blade member and comprises an inner tubular member or sleeve having a proximal length region 36 extending distally from an inner member hub 38, a distal end 40 having a side-facing opening 42 and a flexible or bendable region 44 between proximal length region 36 and distal end 40. The opening 42 communicates with a lumen 46 through the inner member and has a cutting edge 47 comprising a plurality of cutting teeth extending along a peripheral edge of opening 42. When the inner member 18 is disposed within the outer member 16, the inner member extends through the outer member hub 20 with the inner member hub 38 disposed in handpiece 10 proximally of the outer member hub and in driving engagement with a drive shaft of a motor in the handpiece as explained further below. The cutting edge 47 is exposed by the opening 32 and is aligned with or adjacent the opening 32. The flexible region 44 is disposed within or adjacent the bend 24 so that the inner member follows or conforms to the longitudinally or axially bent, curved or angled configuration of the outer member. The proximal length region 36, which is disposed in the proximal length portion 22, is rigid and transmits torque from the motor via the flexible region 44 to rotate the distal end 40 when the inner member 18 is rotated relative to and within the outer member 16. The flexible region 44 allows the inner member 18 to conform to the angled configuration of the outer member 16 as it is rotated relative to and within the outer member. As the inner member is rotated with the outer member, the cutting edge 47 moves past the cutting edge 35, and the cutting edges 35,47 cooperate to cut anatomical tissue adjacent opening 32. As explained further below, anatomical debris may be aspirated from the operative site through a suction passage that may be formed by the lumen 46 of inner member 18, the opening 42 of the inner member forming a suction inlet through which debris is aspirated. Irrigating or flushing fluid may be supplied through or along the instrument 10 to the operative site as also described further below.

The flexible region of the inner member can be formed in various ways, the flexible region 44 being formed by way of example from a reinforced polymeric material as represented by U.S. Pat. No. 5,922,003 to Anctil et al, the entire disclosure of which is incorporated herein by reference. The flexible region may be formed by a helical cut in the inner tubular member and at least one strip of material spirally wound over the helical cut as disclosed in U.S. patent application Ser. No. 09/404,461 filed Sep. 24, 1999, the entire disclosure of which is incorporated herein by reference.

For use as an adenoid blade, which may also be used to perform a tonsillectomy, the distal length portion 26 preferably extends from bend 24 at an angle A of 40 degrees relative to the central longitudinal axis 28, the bend 24 has a radius of curvature of about 0.875 inch and a location about 0.7 inch from the distal end 30, and the opening 32 faces outwardly relative to the direction of the bend, i.e. relative to the center of curvature for the bend, as represented by the RADenoid® Blade of Medtronic Xomed Surgical Products and by the Anctil et al patent previously incorporated herein by reference. As shown in dotted lines in FIG. 1, the opening 32 may face in the direction of the center of curvature for bend 24. A blade in which the opening 32 faces in the direction of the center of curvature for bend 24 and in which the angle A is 40 degrees is represented by the RAD® 40 Curved Blade of Medtronic Xomed Surgical Products, which is particularly useful as a sinus blade in sinus surgery to access the frontal recess in the maxillary sinus, for ethmoid and frontal recess surgery, maxillary polyp removal, uncinectomy and antrostomy, for example. A blade in which the opening 32 faces in the direction of curvature for bend 24 and in which the angle A is 60 degrees is shown in dotted lines in FIG. 1 and is represented by the RAD 60 X-TREME™ Curved Blade of Medtronic Xomed Surgical Products which is also used as a sinus blade. Of course, the blade 12 can be longitudinally or axially straight as shown by dotted lines in FIG. 1.

The powered surgical handpiece 14 comprises a housing 50, a motor 52 disposed in the housing, a battery unit 54 disposed in the housing for powering motor 52 and a control unit 56 externally disposed on the housing for controlling operation of the motor 52. The housing 50 comprises a main body 58 and a cap 60 assembled to the main body to enclose the interior of the handpiece. The main body 58 is tapered at a forward end of the housing, and the cap 60 defines a closed rearward end for the housing. The main body 58, without cap 60 assembled thereto, has an open rearward end providing access to the interior of the housing to facilitate assembly of the instrument and placement of components within the handpiece interior during the assembly process. The housing 50 has an external configuration to be comfortably and securely held in the hand of a surgeon and may have external ridges to facilitate grasping. The housing 50 is made of a medically acceptable material, with the main body 58 and the cap 60 each being formed integrally, unitarily or monolithically as a single piece. As an example, the housing 50 may be made from molded plastic.

As shown in FIG. 4, the forward end of the housing 50 has a longitudinal passage 62 therein extending proximally within the main body 58 from an opening along a forward end wall 64 of the housing. The outer member hub 20 is hollow, with a forward portion 70, a rearward portion 72 and an externally protruding annular flange 74 between the forward and rearward portions. The rearward portion 72 is disposed in passage 62 with a close fit with the flange 74 in abutment with the forward end wall 64 to close off the forward end of the housing 50. Since the battery powered rotary tissue cutting instrument 10 is disposable, the outer member hub 20 may be fixedly secured to the housing 50. The outer member hub may be fixedly secured to the housing in any suitable manner, such as adhesively. The proximal end 76 of the outer member 16 is fixedly secured in the outer member hub 20, and the proximal end 76 may be fixedly secured in the outer member hub in many various ways including adhesively. The proximal end 76 may be externally knurled to facilitate such securement.

The wall of the outer member hub 20 forming forward portion 70 has a hole 78 extending radially therethrough in alignment with an aperture 80 extending radially through the wall of outer member 16. As shown in FIG. 1, an irrigation fitting 82 is mounted in or on the hole 78 and extends from the housing 50 to an open end adapted for removable attachment to an end of an irrigation tube 86. The open end of the irrigation fitting 82 may be barbed to facilitate attachment of the irrigation fitting to the end of the irrigation tube 86, the opposite end of which is connected with a source of irrigating or flushing fluid. The irrigation fitting 82, hole 78 and aperture 80 establish communication between the irrigation tube 86 and the lumen of the outer member 16 for supplying irrigating or flushing fluid to the irrigation passage 87 formed by the small annular or circumferential gap or space between the inside diameter of the outer member 16 and the outside diameter of the inner member 18 as shown in FIG. 4. Fluid supplied to the irrigation passage 87 via the irrigation tube 86 is discharged from the opening 32 and is thusly supplied to the operative site. The instrument 10 is representative of an instrument in which the irrigation fitting is disposed at or near the forward end of the housing 50, but it should be appreciated that the irrigation fitting can be disposed on the instrument at various locations including locations at or near the rearward end of the housing.

The inner member 18 extends proximally from the outer member hub 20 and an annular seal 88 is disposed around the inner member in the outer member hub 20. The seal 88 provides a seal between the outside diameter of inner member 18 and the internal surface of the outer member hub 20 at a location distally of forward end wall 64. The seal 88 prevents the irrigating or flushing fluid from moving proximally there past such that the fluid does not enter the handpiece 14. An aperture 90 in the inner member 18 is located in the handpiece 14 between seal 88 and inner member hub 38. The aperture 90 is located within the outer member hub 20 and, as the inner member 18 is rotated within the outer member 16, the aperture 90 becomes aligned with a hole 92 extending radially through the wall of the outer member hub forming the rearward portion 72. The hole 92 is, in turn, aligned with an opening 94 in the forward end of housing 50. As shown in FIG. 1, a suction fitting 96 is mounted in or on the opening 94 and extends from the housing 50 to an open end adapted for removable attachment to an end of a suction tube 100. An opposite end of the suction tube 100 is connected with a source of suction, and the open end of the suction fitting 96 may be barbed to enhance attachment of the suction fitting to the suction tube 100. The suction fitting 96, opening 94, hole 92 and aperture 90 establish communication between the suction source and the suction passage formed by the lumen 46 of the inner member 18 for aspirating anatomical debris from the operative site via the suction port provided by opening 42 at the distal end of the inner member. The instrument 10 is representative of an instrument in which the suction fitting is disposed at or near the forward end of housing 50. It should be appreciated, however, that the suction fitting can be disposed on the instrument at various locations including locations at or near the rearward end of the housing.

The proximal end of inner member 18 may be fixedly secured in the inner member hub 38, such as adhesively, and the inner member proximal end may be externally knurled to enhance such securement as shown in FIG. 4. A seal and/or bearing 101 may be provided in the passage 62 between outer member hub 20 and inner member hub 38 as also shown in FIG. 4. The inner member hub 38 has a longitudinally extending socket 102 coaxial with the inner member for receiving a drive shaft or pin 104 of motor 52. The drive shaft 104 is thusly coupled in driving engagement with the inner member 18, such that the inner member is rotated by the drive shaft. It should be appreciated that the stationary outer member hub and the rotatably driven inner member hub can have various configurations and sizes depending on the configuration and size of the handpiece and the mounting arrangement by which the hubs are mounted to the handpiece.

The motor 52 is disposed in the housing 50 and is powered by the battery unit 54 to effect rotation of the drive shaft 104 and, therefore, rotation of the inner member 18 relative to and within the outer member 16. Any suitable motor can be used in the present invention including brushed DC motors such as the CERMAC motor RE 280 providing oscillatory rotation at 2800/3400 RPM and the Mabuchi/Aristocraft providing 2500 RPM at eight volts. The battery unit 54 comprises at least one battery electrically connected with the motor 52. Any suitable batteries may be used for the battery unit including conventional dry cell batteries of sufficient voltage to effect rotation of the motor drive shaft at a desired speed for a predetermined amount of use. Accordingly, the battery unit 54 will provide sufficient power to rotate the inner member 18 at the required speed throughout a particular surgical procedure to be performed on a single patient. In particular, the battery unit 54 will provide sufficient power to rotate the inner member at the required speed to ensure completion of an adenoidectomy procedure, a tonsillectomy procedure or a combined adenoidectomy and tonsillectomy (T&A) procedure performed on a single patient. As explained further below, the instrument 10 may be provided with a use-limiting unit whereby the instrument automatically becomes disabled or inoperative subsequent to the predetermined amount of use.

The control unit 56 is disposed along the external surface of the handpiece 14 and comprises one or more switches 57 for effecting instantaneous on/off operation or control of motor 52 for enhanced patient safety. The control unit 56 is operable to selectively allow electric current to flow from the battery unit 54 to the motor 52 to start and stop rotation of the motor drive shaft. The one or more switches 57 may be one or more momentary switches electrically connected with the battery unit 54 or one or more optical coupled switches. The switches 57 may be pressure sensitive switches to be activated via finger pressure and may be provided with an external protective cover or seal 108. The control unit 56 may allow for selection of the drive shaft rotational speed and/or selection of continuous rotation of the drive shaft in one direction or continuous oscillatory rotation of the drive shaft, as well as the selection of other features. As an example, one of the switches 57 shown in FIGS. 1 and 2 may be used to turn the instrument on instantaneously while the other illustrated switch 57 may be used to turn the instrument off instantaneously. As another example, one of the switches 57 may be used for instantaneous on/off operation while the other switch 57 may be used to select either rotational speed or type of rotation, i.e. continuous rotation in one direction or continuous oscillatory rotation in forward and reverse directions.

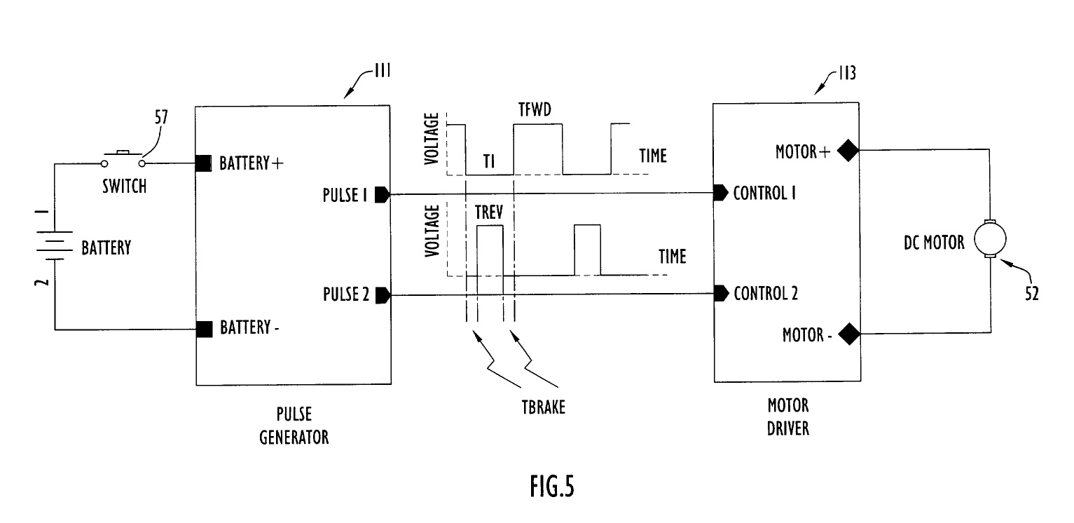

FIG. 5 schematically depicts oscillatory rotational operation of motor 52. Switch 57 includes a momentary switch for instantaneous on/off operation of the instrument 10, and a pulse generator 111 within the handpiece supplies timed pulses of electric current from battery 54 to a motor driver 113, which is also disposed within the handpiece, for rotatably driving the motor 52 in accordance with the pulses supplied by the pulse generator. The pulse generator 111, which may be a phase shifted dual pulse generator with brake, generates two electrical pulses shown as Pulse 1 and Pulse 2 in FIG. 5. FIG. 5 graphically depicts the voltage for Pulse 1 over time, from which it is seen that Pulse 1 is not generated by the pulse generator 111 over time interval T1 and is generated by the pulse generator at a predetermined constant voltage over time interval TFWD. As graphically depicted in FIG. 5 for Pulse 2, Pulse 2 is not generated by the pulse generator 111 while Pulse 1 is generated over time interval TFWD. Also, Pulse 2 is not generated over first and second time intervals TBRAKE at the beginning and end, respectively, of time interval T1. Pulse 2 is generated by the pulse generator 111 at the predetermined constant voltage over time interval TREV, between first and second time intervals TBRAKE and corresponding to the time interval T1 minus the first and second time intervals TBRAKE during which time Pulse 1 is not generated. It should be appreciated that various timing circuits can be incorporated in the handpiece to control or modulate the pulses in any desired manner.

Motor driver 113 may be a brushed DC motor driver for controlling rotation of the drive shaft of motor 52 in response to Pulse 1 and Pulse 2. Motor driver 113 may include appropriate circuitry in which Control 1 of motor driver 113 causes forward, i.e. clockwise, rotation of the motor drive shaft over time interval TFWD in response to generation of Pulse 1 by the pulse generator 111. When Pulse 1 ceases to be generated at the end of time interval TFWD, first time interval TBRAKE takes effect, allowing forward rotation of the motor drive shaft to slow down or stop since neither Pulse 1 nor Pulse 2 is generated. At the conclusion of the first TBRAKE time interval, Pulse 2 is generated by the pulse generator 111, and Control 2 of the motor driver 113 causes reverse, i.e. counterclockwise, rotation of the motor drive shaft over time interval TREV. At the conclusion of time interval TREV, the second time interval TBRAKE takes effect and neither Pulse 1 nor Pulse 2 is generated while reverse rotation of the drive shaft slows down or stops. At the conclusion of the second TBRAKE time interval, time interval TFWD again commences and the motor driver 113 causes forward rotation of the motor drive shaft. In this manner, the drive shaft 104 and, therefore, the inner member 18, is continuously oscillatorily rotated in alternating forward and reverse directions for amplified cutting action by the inner member cutting edge 47 moving past the outer member cutting edge 35. Of course, the motor drive shaft can be continuously rotated in one direction only by supplying an appropriate continuous voltage or current to the motor 52 using conventional electronic components and circuitry.

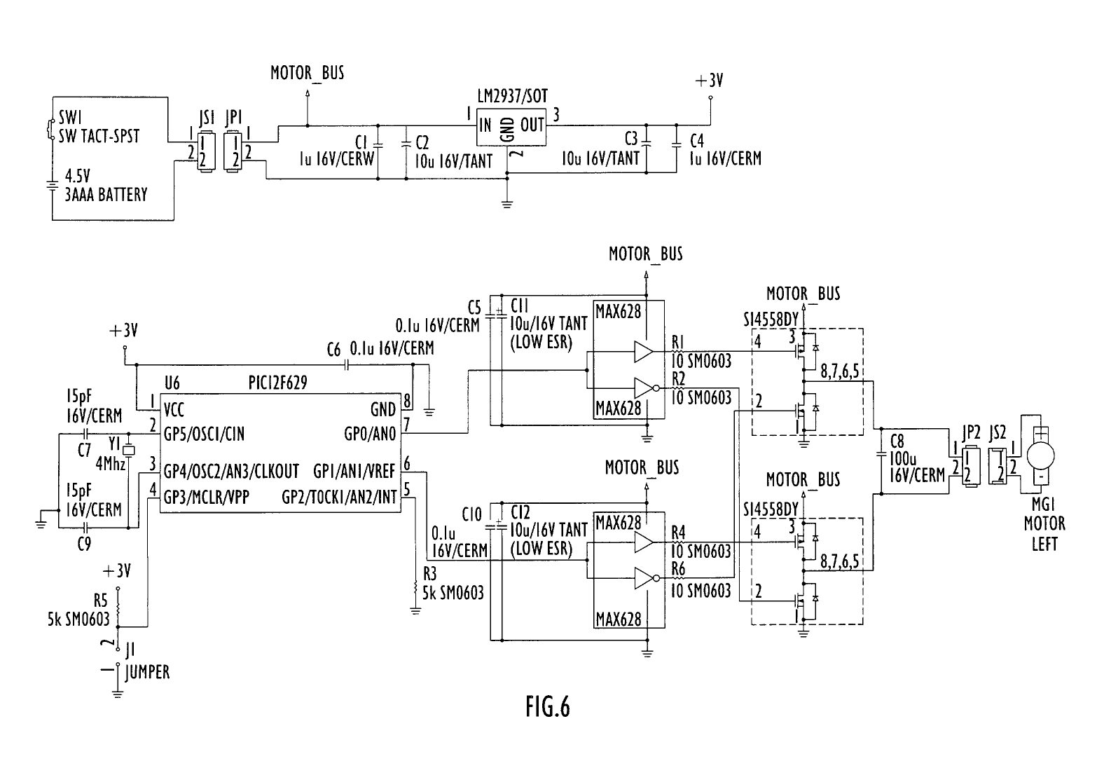

FIG. 6 depicts a representative motor drive circuit for the powered surgical handpiece 14. In the representative circuit, a crystal controlled programmed microprocessor outputs a timed pulse driving the motor control circuitry. The pulses are timeD and coordinated to drive the motor, through motor drivers, first in one direction and then reversed. This cycle is repeated as long as the circuitry is active. The program for the microprocessor can be modified to allow single direction control of the motor. The timing pulses have the ability of being programmed as required. It should be appreciated that other electrical components and circuitry may be used to obtain continuous rotation of the motor drive shaft in one direction or to obtain continuous oscillatory rotation of the motor drive shaft in forward and reverse directions, and that the representative circuit should be considered illustrative and not limiting.

The disposable battery powered rotary tissue cutting instrument 10 has a use-limiting unit 123, disposed in handpiece 14, by which the instrument 10 becomes disabled and cannot be operated after a predetermined amount of use. The use-limiting unit 123 is depicted in flow chart form in FIG. 7 and includes electrical components and circuitry for determining or measuring a predetermined amount of use, for generating a disabling signal in response to the occurrence of the predetermined amount of use, and for disabling the instrument in response to the disabling signal by preventing electric current from being supplied to the motor from the battery unit. The predetermined amount of use before which the instrument 10 becomes disabled or inoperative is selected to ensure that the instrument does not become inoperative or disabled prior to completion of a surgical procedure to be performed on a single patient using the instrument. The predetermined amount of use may, for example, be measured in terms of a predetermined amount of time to ensure completion of the surgical procedure, and the handpiece 14 may contain one or more conventional timers and/or electrical circuits for generating a disabling signal upon expiration of the predetermined amount of time. As another example, the predetermined amount of use may be measured in terms of battery voltage, in which case the handpiece 14 may include one or more conventional electrical components and/or circuits for measuring the open circuit voltage of the one or more batteries and for generating the disabling signal when the battery voltage falls below a predetermined value. The disabling signal operates to open the motor drive circuit and/or to cause an excessive battery drain. In the motor drive circuit of FIG. 6, for example, the disabling signal may operate to permanently deactivate an appropriate one or more of the transistors Q1-Q5 to open the motor drive circuit. As another example, the disabling signal may operate to drain the battery power, such as to drain the battery current into a resistor for dissipation as heat.

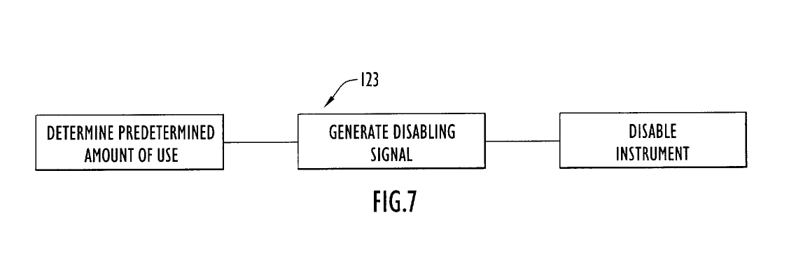

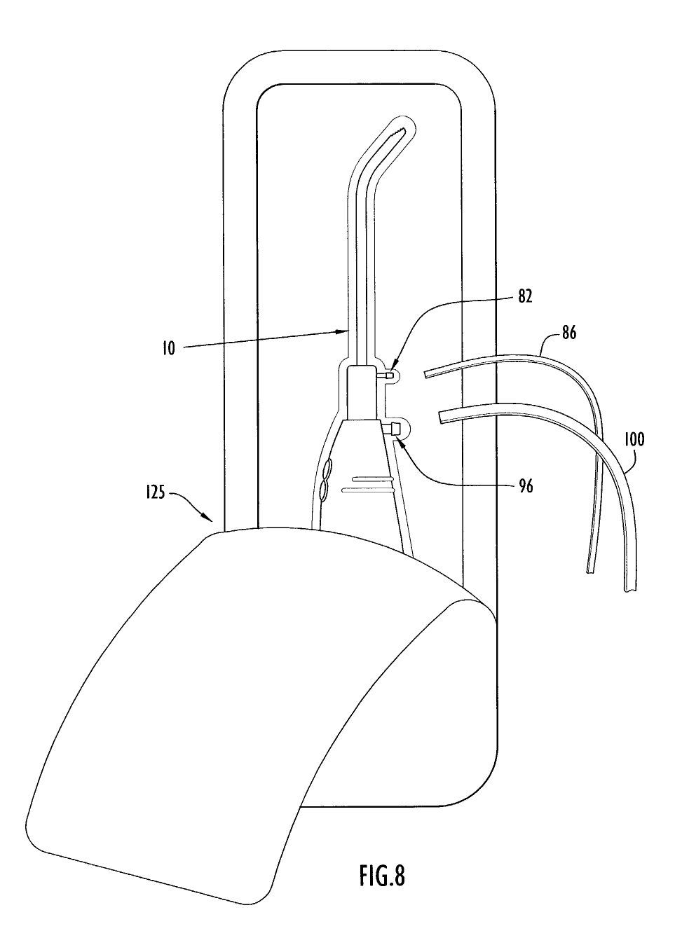

The disposable battery powered rotary tissue cutting instrument 10 is used during a surgical procedure performed on a single patient to remove soft anatomical tissue at an operative site in the patient's body. The instrument 10 is preferably provided in medically sterile condition contained within a sterile sealed package 125 as shown in FIG. 8. Preparatory to a surgical procedure to be performed using the disposable battery powered rotary tissue cutting instrument 10, the instrument is removed from the package 125 as shown in FIG. 8 and, if irrigation and/or aspiration are to be employed during the surgical procedure, the irrigation tube 86 is attached to the irrigation fitting 82 and/or the suction tube 100 is attached to the suction fitting 96 as also shown in FIG. 8. Accordingly, in just a few simple steps, the disposable battery powered rotary tissue cutting instrument 10 is ready for use in the surgical procedure.

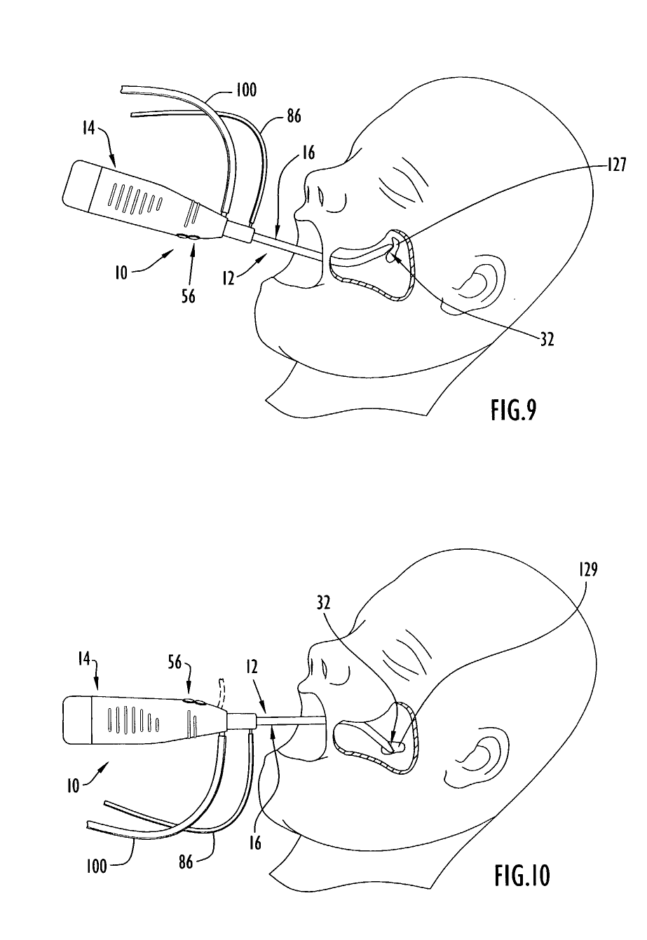

FIG. 9 illustrates an adenoidectomy procedure performed using the thusly prepared disposable battery powered rotary tissue cutting instrument 10. The distal end of the blade 12 is introduced through the patient's mouth and into the nasopharynx with the opening 32 in the outer member 16 facing and adjacent an adenoid 127. The handpiece 14 is oriented with the control unit 56 at the bottom and the irrigation and suction tubes 86 and 100 at the top thereof so that the blade 12 is angled upwardly to facilitate access to the adenoid 127. The control unit 56 is conveniently located on the handpiece 14 to be easily accessed by a finger of the surgeon's hand grasping the handpiece regardless of whether the control unit 56 is facing downwardly as shown in FIG. 9 or upwardly as shown in FIG. 10 depending on the orientation of the handpiece. As described above, the control unit 56 may be manually set by the surgeon to obtain a desired rotational speed and/or a desired type of rotation, i.e. continuous rotation in one direction or continuous oscillatory rotation in forward and reverse directions, and the control unit may be manually set via finger pressure applied to the appropriate switch or switches along the external surface of the handpiece. To activate the instrument, the control unit 56 is manually engaged by a finger of the surgeon's hand, such as by pressing the appropriate switch along the external surface of the handpiece, to cause electric current to flow to the motor from the battery unit. Instantaneously, the inner member 18 begins rotating within the outer member 16 and, depending on the design of the handpiece and/or the operational selection made by the surgeon, the inner member 18 is rotated within the outer member 16 either continuously in one direction or continuously oscillatorily in alternating forward and reverse directions. The inner member cutting edge 47 moves past the outer member cutting edge 35 to remove, cut or shave adenoid tissue positioned adjacent the opening 32. The adenoidectomy is initiated in the anterior nasopharynx, near the choana. Removal of adenoid tissue is initiated using a light touch with the tissue cutting blade, with tissue removal being accomplished by moving the blade side to side and/or sweeping the blade anterior to posterior. Sweeping the blade anteriorly to posteriorly is particularly effective along the torus tubarias. The cutting blade allows a precise progressive removal of adenoid tissue while simultaneous suction or aspiration via opening 42, lumen 46 and suction tube 100 allows blood and tissue to be evacuated from the operative site for enhanced visualization. Tissue at the superior choana and along the torus tubarias can be shaved away to effect a more thorough adenoidectomy which minimizes the potential for tissue regrowth and symptom recurrence. Continuous suction through the inner member 18 keeps the surgical field visible while tissue is removed from difficult areas such as the superior choana and along the posterior torus tubarias. The precise shaving action of the cutting blade removes well-defined portions of tissue exactly where the blade is placed. The increased surgical accuracy allows easy removal of hypertrophic adenoid tissue from the posterior nasal cavity and along the torus. Upon removal of a sufficient amount of tissue, rotation of the inner member is stopped by terminating the flow of electric current from the battery unit to the motor via manual engagement of the control unit 56 by a finger of the surgeon's hand. The cutting blade may be used to cauterize tissue, typically in the area where tissue has been removed using the cutting blade, where the instrument 10 is provided with a wire 15, shown in dotted lines in FIG. 10, electrically coupling the outer member with a source of electricity as described further below for instrument 210. Accordingly, FIG. 10 is representative of the steps of removing adenoid tissue by cutting or resecting the adenoid tissue using the cutting blade, thereafter contacting the distal end of the outer member with adenoid tissue in the area from which tissue was removed, and supplying electric current to the distal end of the outer member to thereby cauterize the tissue.

The handpiece 14 is designed to provide sufficient power to rotate the inner member as much as needed throughout the entire surgical procedure since the predetermined amount of use ensures that the instrument 10 will remain operative throughout the surgical procedure. Upon completion of the adenoidectomy, the blade 12 is withdrawn from the patient's mouth, and the instrument 10 is disposed of as an integral unit with the blade 12 affixed to the handpiece 14. The irrigation tube 86 and the suction tube 100 can remain attached to the handpiece for disposal therewith, thereby simplifying and reducing the time required for post-operative clean-up. Where provided, the use-limiting unit 123 prevents reuse of the instrument 10 in another surgical procedure in the event that the instrument is not properly disposed of. Subsequent to completion of the adenoidectomy and prior to any opportunity for the instrument 10 to be used in another surgical procedure performed on a different patient, the use-limiting unit 123 operates to render the instrument 10 disabled or inoperative so that the instrument cannot be reused in the event that it has not already been disposed of.

The disposable battery powered rotary tissue cutting instrument 10 may also be used to perform a tonsillectomy procedure as shown in FIG. 10, and the tonsillectomy procedure may be performed separately from or in conjunction or combination with the adenoidectomy procedure. To perform the tonsillectomy procedure, the distal end of the blade 12 is inserted in the patient's mouth and the opening 32 in the outer member 16 is positioned adjacent a tonsil 129. The handpiece 14 is inverted from the position shown in FIG. 9 so that the blade 12 is angled downwardly to facilitate access to the tonsil 129 and proper positioning of opening 32. The control unit 56 is used to effect rotation of the inner member 18 within the outer member 16 as described above for the adenoidectomy procedure. The cutting edges 35,47 effect removal of anatomical tissue of the tonsil while suction or aspiration is effected through the inner member. The control unit 56 is operated to stop the outer member from rotating upon sufficient removal of tissue and, upon completion of the tonsillectomy, the blade 12 is withdrawn from the patient's mouth. The instrument 10 is then disposed of prior to or subsequent to becoming automatically disabled or inoperative due to the use-limiting unit 123.

An alternative disposable battery powered rotary tissue cutting instrument is illustrated at 210 in FIG. 11. The disposable battery powered rotary tissue cutting instrument 210 comprises a tissue cutting blade 212 attached to a powered surgical handpiece 214. The blade 212 is similar to blade 12 and includes an elongate outer tubular member 216 and an elongate inner member (not visible in FIG. 11) rotatably disposed within the outer member. The blade 212 is different from blade 12 in that the outer and inner members for blade 212 are longitudinally or axially straight. Accordingly, the outer member 216 extends distally from the handpiece 214 to distal end 230 having side-facing opening 232 and cutting edge 235 comprising a plurality of cutting teeth extending along a peripheral edge of opening 232. The outer member 212 is rigidly or fixedly mounted to the handpiece 214 while the inner member is rotatably mounted to the handpiece 214 for rotation within the outer member.

The powered surgical handpiece 214 comprises housing 250, motor 252 disposed in the housing, battery unit 254 disposed in the housing for powering motor 252 and control unit 256 externally disposed on the housing for controlling operation of the motor 252. The housing 250 comprises a main body 258 and a cap 260 assembled to the main body to enclose the interior of the housing. The main body 258, without cap 260 assembled thereto as shown in FIG. 11, has an open rearward end providing access to the interior of the housing to facilitate assembly of the instrument and placement of components within the housing interior. FIG. 11 illustrates the battery unit 254 as comprising two batteries 255 arranged within the housing in end-to-end relation. The control unit 256 for instrument 210 is depicted as comprising a sliding switch 257 movable or slidable within a longitudinal recess 259 in the housing 250. The switch 257 can be moved to one or more discrete positions along recess 259 corresponding to one or more different functional selections for the instrument 210 such as drive shaft rotational speed, continuous rotation of the drive shaft in one direction, continuous oscillatory rotation of the drive shaft, and on/off operation. The housing 250 has an external configuration different from the external configuration of housing 250, but the external configuration of housing 250 is also designed to be comfortably and securely held in the hand of a surgeon. The handpiece 214 is representative of a handpiece in which the suction fitting is located at or near the rearward end of the housing. The suction fitting 296 for handpiece 214 is located at or near the rearward end of the housing 250 and is located proximally of the main body 258. The suction fitting 296 is disposed on an end of a suction conduit 297, and at least part of the suction conduit 297 extends externally alongside the housing 250. The suction conduit 297 extends rearwardly alongside the housing 250 parallel with a central longitudinal axis of the instrument 210 to the end of the suction conduit which is angled outwardly from the housing 250 to the suction fitting 296. As shown in FIG. 11, the housing 250 may be provided with a groove for accommodating the suction conduit 297.

The disposable battery powered rotary tissue cutting instrument 210 is further representative of an instrument in which the blade may be used for electric cautery. The disposable battery powered rotary tissue cutting instrument 210 includes an electrically conductive wire 215 having an end connected to the outer member 216, typically being connected to the proximal end of the outer member 216, and an opposite end connected to a source of electricity, such as an electrosurgical generator (not shown), in order to supply electric current to the outer member 216. When the electrosurgical generator is activated to supply current to outer member 216, the distal end 230 may be placed in contact with anatomical tissue to effect cauterization of the tissue. An insulative tubular sheath is concentrically disposed over the outer member 216, the sheath 217 extending distally from the handpiece 214 to terminate a small distance proximally of the cutting edge 235. Accordingly, the distal end 230 and cutting edge 235 remain exposed from the sheath, allowing the distal end to be used to cauterize anatomical tissue while the sheath 217 protects surrounding anatomical tissue from contacting the outer member 216. It should thusly be appreciated that in the methods described above, the cutting blade of the instrument can be used to cut or resect anatomical tissue and can also be used to cauterize anatomical tissue in the patient's body. Typically, the cutting blade will be used to cut or resect anatomical tissue and will thereafter be used to cauterize anatomical tissue in the area of the patient's body from which the tissue was resected.

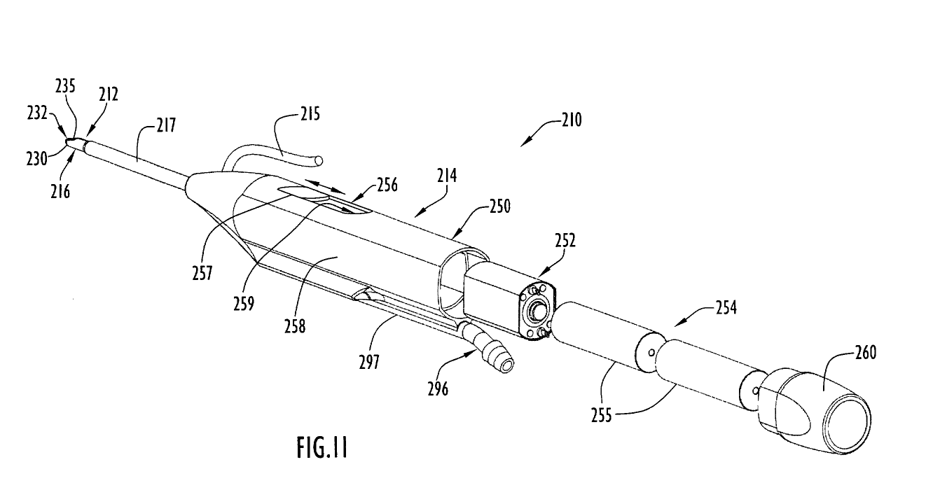

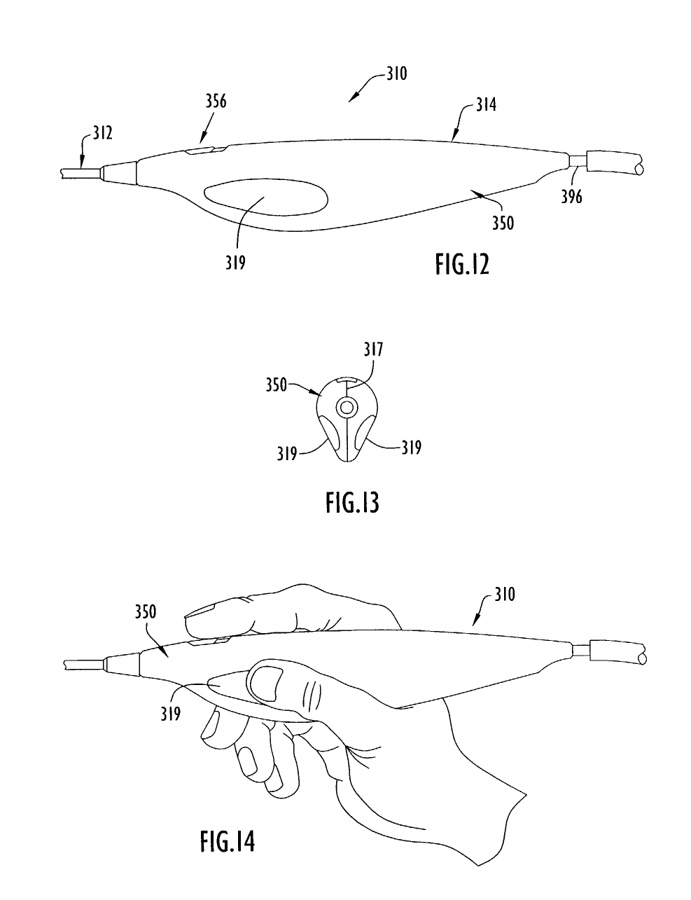

FIGS. 12-14 illustrate a further alternative disposable battery powered rotary tissue cutting instrument 310. The instrument 310 comprises blade 312 and handpiece 314, with the blade 312 being similar to blade 212. The blade 312 is shown in FIGS. 12 and 14 but is not shown in FIG. 13 which illustrates the housing 350 of handpiece 314 without the blade assembled thereto. The housing 350 is split longitudinally to comprise two separate pieces or parts for assembly of components therein. The parts are secured to abut one another along a longitudinal seam 317, shown in FIG. 13, when the housing 350 is assembled as shown in FIGS. 12-14. The handpiece 314 has a suction fitting 396 forming the rearward end of the housing 350. The housing 350 has upper and lower surfaces with an outward curvature between the forward and rearward ends of the housing, the forward and rearward ends being tapered in cross-section. A central portion of housing 350 between the tapered forward and rearward ends has a cross-sectional configuration similar to an inverted teardrop shape. Opposing sides of the central portion have distinctive finger engaging areas 319 located thereon near the lower surface of the housing. When the handpiece 314 is held by the hand of a surgeon as shown in FIG. 14, the thumb of the hand is engaged with the finger engaging area 319 on one side of the handpiece while the middle finger of the hand extends over the finger engaging area 319 on the opposite side of the handpiece. The finger engaging areas 319 may each be formed as a frictional or a non-sliding surface formed by recesses, textured surfaces and/or frictional materials of various types. The finger engaging areas are preferably sufficiently large in size to accommodate various hand sizes and grasping styles. The configuration of handpiece 314 and the finger engaging areas 319 ensure that the handpiece remains stable when grasped in the hand of the surgeon so that the handpiece does not inadvertently slide, rotate or otherwise move. Stability of the handpiece in the grasping hand provides enhanced accuracy in manipulating and positioning of the blade at the operative site.

The disposable battery powered rotary tissue cutting instruments of the present invention can be provided with either or both of an irrigation passage and an aspiration passage. The irrigation passage can be located internally through the instruments or externally along the instruments, and the aspiration passage can be located internally through the instruments or externally along the instruments. Illustrative arrangements for irrigation and aspiration are represented by U.S. Pat. No. 5,782,795 to Bays, U.S. Pat. No. 5,916,231 to Bays and U.S. Pat. No. 6,312,438 B1 to Adams, the entire disclosures of which are incorporated herein by reference.

The powered surgical handpieces of the present invention can be designed to receive standard tissue cutting blades. Various types of motors can be used in the handpieces, and various voltages can be used depending on the speed desired for the motor drive shaft. For typical surgical applications, the motor drive shaft may be designed to rotate at 2000-8600 RPM. The battery units may comprise one or more batteries arranged in the handpieces in many various ways. The control units can include various switches for on/off operation, speed selection, rotational selection and/or other features. The control units are preferably sealed so that substances cannot enter the handpieces and degrade the mechanical and electrical components therein. The control units are designed to effect instantaneous on/off operational control to minimize patient risk and surgical error. Various conventional electrical components and circuits can be incorporated in the handpieces to achieve various desired operational parameters and functions. The present invention allows a reduction in operating room equipment and set-up for surgical procedures and particularly for surgical procedures of relatively short duration such as adenoidectomies, tonsillectomies and combined tonsillectomy and adenoidectomy (T&A) procedures. The instruments may be used in various procedures of the ears, nose and/or throat requiring soft tissue removal including sinus and laryngeal procedures. The need to sterilize the instruments for repeated use is eliminated in that the instruments are disposed of after single patient use. The motor and battery unit do not add significantly to the weight of the handpieces, allowing the handpieces to be manipulated by a surgeon with precision and accuracy. The handpieces are not connected to any heavy power cords or cables thereby enhancing the precision and accuracy with which the handpieces and, therefore, the cutting blades, can be manipulated. The instruments can be economically manufactured for single patient use without the need for costly design features and components needed to render the instruments tolerant to repeated sterilizations. Single patient use is ensured due to the use-limiting unit which permits only a predetermined amount of use of the instruments, and the predetermined amount of use does not allow the instruments to be reused on a different patient.

Inasmuch as the present invention is subject to many variations, modifications and changes in detail, it is intended that all subject matter discussed above or shown in the accompanying drawings be interpreted as illustrative only and not be taken in a limiting sense.

What is claimed is:

1. A disposable battery powered rotary tissue cutting instrument for soft tissue removal comprising
a blade comprising an elongate tubular outer member and an elongate tubular inner member rotatably disposed within said outer member, said outer member extending distally from an outer member hub to a distal end having a side-facing opening, said outer member having a cutting edge extending along a peripheral edge of said opening, said inner member extending distally from an inner member hub to a distal end having a side-facing opening adjacent said opening in said outer member, said inner member including a cutting edge extending along a peripheral edge of said opening in said inner member and cooperable with said cutting edge of said outer member to cut soft anatomical tissue adjacent said opening in said outer member when said inner member is rotated relative to and within said outer member; and
a powered surgical handpiece comprising a housing fixedly mounting said outer member hub and rotatably mounting said inner member hub, a motor within said housing for rotating said inner member, a battery unit within said housing for powering said motor, and a control unit disposed along an external surface of said housing for controlling operation of said motor, said motor having a drive shaft in driving engagement with said inner member hub, said battery unit including at least one battery electrically connected with said motor, said control unit being electrically connected with said battery unit and being operable to selectively allow electric current to flow from said at least one battery to said motor to start and stop rotation of said drive shaft, said instrument being disposable upon completion of a surgical procedure performed with said instrument on a single patient.

2. The disposable battery powered rotary tissue cutting instrument recited in claim 1 wherein said outer member includes a longitudinally straight proximal length portion extending distally from said outer member hub to a bend and a longitudinally straight distal length portion extending from said bend to said distal end of said outer member, and said inner member is flexible to conform to the configuration of said outer member as said inner member is rotated by said drive shaft relative to and within said outer member.

3. The disposable battery powered rotary tissue cutting instrument recited in claim 2 wherein said opening in said outer member faces in a direction away from the center of curvature for said bend.

4. The disposable battery powered rotary tissue cutting instrument recited in claim 2 wherein said opening in said outer member faces in the direction of the center of curvature for said bend.

5. The disposable battery powered rotary tissue cutting instrument recited in claim 3 wherein said distal length portion defines an angle of 40 degrees with said proximal length portion.

6. The disposable battery powered rotary tissue cutting instrument recited in claim 4 wherein said distal length portion defines an angle of 40 degrees with said proximal length portion.

7. The disposable battery powered rotary tissue cutting instrument recited in claim 4 wherein said distal length portion defines an angle of 60 degrees with said proximal length portion.

8. The disposable battery powered rotary tissue cutting instrument recited in claim 1 wherein each of said cutting edges comprises a plurality of cutting teeth.

9. The disposable battery powered rotary tissue cutting instrument recited in claim 1 wherein said housing comprises a main body defining a forward end of said housing receiving said outer member and said inner member therethrough and a cap assembled to said main body and defining a closed rearward end of said housing.

10. The disposable battery powered rotary tissue cutting instrument recited in claim 9 wherein said main body includes a forward end wall and said outer member hub abuts said forward end wall to close said forward end of said housing.

11. The disposable battery powered tissue cutting instrument recited in claim 9 wherein each of said main body and said cap are integrally, unitarily formed of a medically acceptable material.

12. The disposable battery powered tissue cutting instrument recited in claim 1 wherein said housing comprises two separate parts secured in abutment with one another along a longitudinal seam.

13. The disposable battery powered tissue cutting instrument recited in claim 1 wherein said handpiece includes finger engaging areas on opposite sides of said housing.

14. The disposable battery powered rotary tissue cutting instrument recited in claim 1 wherein said motor includes a brushed DC motor.

15. The disposable battery powered rotary tissue cutting instrument recited in claim 1 wherein said control unit is adapted to start and stop rotation of said drive shaft instantaneously.

16. The disposable battery powered rotary tissue cutting instrument recited in claim 1 wherein said control unit is sealed along said external surface of said housing.

17. The disposable battery powered rotary tissue cutting instrument recited in claim 15 wherein said control unit includes at least one momentary switch.

18. The disposable battery powered rotary tissue cutting instrument recited in claim 15 wherein said control unit includes at least one optical coupled switch.

19. The disposable battery powered rotary tissue cutting instrument recited in claim 1 wherein said outer member has an aperture therein communicating with an irrigation fitting attachable to an irrigation tube connected with a source of irrigating fluid, said aperture communicating with an irrigation passage defined by a circumferential gap between said outer member and said inner member to allow the irrigating fluid to be discharged via said opening in said outer member.

20. The disposable battery powered rotary tissue cutting instrument recited in claim 19 wherein said irrigation fitting is located near a forward end of said housing.

21. The disposable battery powered rotary tissue cutting instrument recited in claim 20 wherein said irrigation fitting is located on said outer member hub.

22. The disposable battery powered rotary tissue cutting instrument recited in claim 1 wherein said housing includes a suction fitting for attachment to a suction tube connected with a source of suction and said inner member has an aperture therein that communicates with said suction fitting, said aperture in said inner member communicating with a suction passage defined by a lumen through said inner member to aspirate anatomical debris through said opening in said inner member.

23. The disposable battery powered rotary tissue cutting instrument recited in claim 22 wherein said suction fitting is located near a rearward end of said housing.

24. The disposable battery powered rotary tissue cutting instrument recited in claim 22 wherein said suction fitting is located near a forward end of said housing.

25. The disposable battery powered rotary tissue cutting instrument recited in claim 22 wherein said outer member has an aperture therein communicating with an irrigation fitting attachable to an irrigation tube connected with a source of irrigating fluid, said aperture communicating with an irrigation passage defined by a circumferential gap between said outer member and said inner member to allow the irrigating fluid to be discharged via said opening in said outer member.

26. The disposable battery powered rotary tissue cutting instrument recited in claim 25 wherein said aperture in said inner member is disposed proximally of said aperture in said outer member and further including a seal in said outer member hub between said aperture in said inner member and said aperture in said outer member to prevent the irrigating fluid from moving proximally past said seal.

27. The disposable battery powered rotary tissue cutting instrument recited in claim 1 and further including an electrical circuit in said housing for supplying the electric current to said motor to rotate said drive shaft continuously in one direction.

28. The disposable battery powered rotary tissue cutting instrument recited in claim 1 and further including an electrical circuit in said housing for supplying the electric current to said motor to rotate said drive shaft continuously oscillatorily in forward and reverse directions.

29. The disposable battery powered rotary tissue cutting instrument recited in claim 1 wherein said instrument further includes a wire for electrically coupling said outer member to an electrical source for electrifying said outer member and further including an insulative sheath over said outer member extending from said handpiece to terminate at an edge beyond which said distal end of said outer member is exposed.

30. A disposable battery powered rotary tissue cutting instrument for removal of anatomical tissue comprising
a blade comprising an elongate tubular outer member and an elongate inner member rotatably disposed within said outer member, said outer member extending distally from a proximal end to a distal end having an opening, said inner member extending distally from a proximal end to a distal end having a cutting edge exposed by said opening for cutting anatomical tissue when said inner member is rotated within said outer member; and
a powered surgical handpiece comprising a housing fixedly mounting said proximal end of said outer member and rotatably mounting said proximal end of said inner member, a motor disposed within said housing and having a drive shaft coupled in driving engagement with said proximal end of said inner member, a battery unit in said housing for supplying electric current to said motor to rotate said drive shaft, and a use-limiting unit in said housing for automatically preventing the electric current from being supplied to said motor after a predetermined amount of use of said instrument, whereby said instrument is automatically disabled subsequent to a surgical procedure performed with said instrument on a single patient.

31. The disposable battery powered rotary tissue cutting instrument recited in claim 30 wherein said handpiece includes an electrical circuit by which the electric current is supplied to said motor from said battery unit and said use-limiting unit opens said circuit to disable said instrument.

32. The disposable battery powered rotary tissue cutting instrument recited in claim 30 wherein said battery unit includes at least one battery and said use-limiting unit causes electric current to drain from said battery.

33. A disposable battery powered rotary tissue cutting instrument for removal of anatomical tissue comprising
a blade comprising an elongate tubular outer member and an elongate inner member rotatably disposed within said outer member, said outer member extending distally from a proximal end to a distal end having an opening, said inner member extending distally from a proximal end to a distal end having a cutting edge exposed by said opening for cutting anatomical tissue when said inner member is rotated within said outer member; and
a powered surgical handpiece comprising a housing fixedly mounting said proximal end of said outer member and rotatably mounting said proximal end of said inner member, a motor disposed within said housing and having a drive shaft coupled in driving engagement with said proximal end of said inner member, a battery unit in said housing for supplying electric current to said motor to rotate said drive shaft, and a pulse generator in said housing for supplying the electric current from said battery unit to said motor in timed pulses to rotate said drive shaft continuously oscillatorily in forward and rearward directions, said instrument being disposable upon completion of a surgical procedure performed with said instrument on a single patient.

34. The disposable battery powered rotary tissue cutting instrument recited in claim 33 wherein said pulse generator alternatingly supplies first and second electrical pulses to said motor for rotation of said drive shaft in the forward and reverse directions, respectively.

35. The disposable battery powered rotary tissue cutting instrument recited in claim 34 wherein said pulse generator allows said drive shaft to brake between said first and second pulses.

36. A method of performing a surgical procedure on a patient comprising the steps of
grasping a surgical handpiece of a sterile rotary tissue cutting instrument comprising an elongate tubular outer member extending distally from a proximal end fixedly mounted to the handpiece and an elongate inner member rotatably disposed in the outer member and extending distally from a proximal end coupled in driving engagement with a drive shaft of a motor disposed in the handpiece;
positioning a distal end of the outer member at an operative site in the patient's body such that an opening in the distal end of the outer member is positioned adjacent anatomical tissue to be removed;
causing electric current to flow to the motor from a battery unit within the handpiece;
continuously rotating the drive shaft to rotate the inner member relative to and within the outer member in response to the flow of electric current to the motor;
cutting the anatomical tissue adjacent the opening with a cutting edge of the inner member that is exposed from the opening as the inner member is continuously rotated within the outer member;
terminating the flow of electric current from the battery to the motor;
stopping rotation of the drive shaft to stop rotation of the inner member in response to the termination of electric current flow to the motor;
withdrawing the instrument from the patient's body; and
automatically disabling the instrument subsequent to completion of the surgical procedure on the patient such that the instrument cannot be reused.

37. The method of performing a surgical procedure recited in claim 36 wherein said steps of causing and terminating include applying finger pressure to a control unit along an external surface of the handpiece.

38. The method of performing a surgical procedure recited in claim 37 wherein said step of continuously rotating includes starting continuous rotation of the drive shaft instantaneously in response to said step of causing and said step of stopping includes stopping rotation of the drive shaft instantaneously in response to said step of terminating.

39. The method of performing a surgical procedure recited in claim 36 wherein said step of automatically disabling includes preventing the flow of electric current from the battery to the motor.

40. The method of performing a surgical procedure recited in claim 39 wherein said step of automatically disabling includes opening an electrical circuit within the handpiece so that electric current cannot flow to the motor from the battery.

41. The method of performing a surgical procedure recited in claim 39 wherein said step of automatically disabling includes draining electric current from the battery.

42. The method of performing a surgical procedure recited in claim 36 wherein said step of automatically disabling includes disabling the instrument after a predetermined amount of use.

43. The method of performing a surgical procedure recited in claim 36 wherein said step of continuously rotating includes continuously rotating the drive shaft in one direction.

44. The method of performing a surgical procedure recited in claim 36 wherein said step of continuously rotating includes continuously oscillatorily rotating the drive shaft in forward and reverse directions.

45. The method of performing a surgical procedure recited in claim 36 and further including, subsequent to said step of stopping and prior to said step of withdrawing, the steps of contacting anatomical tissue at the operative site with the distal end of the outer member and supplying electric current to the distal end of the outer member to cauterize the tissue.

46. A method of performing a surgical procedure on a patient including an adenoidectomy comprising the steps of
introducing a distal end of a blade of a rotary tissue cutting instrument in the patient's nasopharynx, the blade comprising an elongate tubular outer member and an elongate tubular inner member rotatably disposed in the outer member, the outer member having a proximal end fixedly secured to a powered surgical handpiece of the rotary tissue cutting instrument, a distal end having a side-facing opening, and a cutting edge extending along a peripheral edge of the opening, the inner member having a proximal end coupled with a drive shaft of a motor disposed in the handpiece, a distal end with a side-facing opening adjacent the opening in the outer member and a cutting edge extending along a peripheral edge of the opening in the inner member;
positioning the opening in the outer member adjacent an adenoid;
causing electric current to flow to the motor from a battery unit within the handpiece;
continuously rotating the drive shaft to rotate the inner member relative to and within the outer member in response to the flow of electric current to the motor;
cutting anatomical tissue of the adenoid as the cutting edge of the inner member moves past the cutting edge of the outer member as the inner member is rotated within the outer member;
terminating the flow of electric current from the battery unit to the motor;
stopping rotation of the drive shaft to stop rotation of the inner member in response to the termination of electric current flow to the motor;
withdrawing the blade from the nasopharynx; and
disposing of the instrument upon completion of the surgical procedure on the patient.

47. The method of performing a surgical procedure recited in claim 46 wherein said steps of causing and terminating include applying finger pressure to a control unit along an external surface of the handpiece.

48. The method of performing a surgical procedure recited in claim 47 wherein said step of continuously rotating includes starting continuous rotation of the drive shaft instantaneously in response to said step of causing and said step of stopping includes stopping rotation of the drive shaft instantaneously in response to said step of terminating.

49. The method of performing a surgical procedure recited in claim 46 wherein said step of continuously rotating includes continuously rotating the drive shaft in one direction.

50. The method of performing a surgical procedure recited in claim 46 wherein said step of continuously rotating includes continuously oscillatorily rotating the drive shaft in forward and reverse directions.

51. The method of performing a surgical procedure recited in claim 46 and further including, subsequent to said step of withdrawing and prior to said step of disposing, performing a tonsillectomy on the patient comprising the steps of introducing the distal end of the blade through the patient's mouth to position the opening in the outer member adjacent a tonsil, causing electric current to flow to the motor from the battery unit, continuously rotating the drive shaft to rotate the inner member relative to and within the outer member in response to the flow of electric current to the motor, cutting anatomical tissue of the tonsil as the cutting edge of the inner member moves past the cutting edge of the outer member as the inner member is rotated within the outer member, terminating the flow of electric current from the battery unit to the motor, stopping rotation of the drive shaft to stop rotation of the inner member in response to the termination of electric current flow to the motor, and withdrawing the blade from the patient's mouth.

52. The method of performing a surgical procedure recited in claim 46 and further including, subsequent to said step of withdrawing, the step of automatically disabling the instrument.

53. The method of performing a surgical procedure recited in claim 51 and further including, subsequent to said step of withdrawing the blade from the patient's mouth upon completion of the tonsillectomy, the step of automatically disabling the instrument.

54. The method of performing a surgical procedure recited in claim 46 and further including, subsequent to said step of stopping and prior to said step of withdrawing, the steps of contacting the distal end of the outer member with anatomical tissue in the area where tissue of the adenoid was cut and supplying electric current to the distal end of the outer member to cauterize the anatomical tissue in contact with the distal end of the outer member.

55. A method of performing a surgical procedure on a patient including a tonsillectomy comprising the steps of
introducing a distal end of a blade of a rotary tissue cutting instrument in the patient's mouth, the blade comprising an elongate tubular outer member and an elongate tubular inner member rotatably disposed in the outer member, the outer member having a proximal end fixedly secured to a powered surgical handpiece of the rotary tissue cutting instrument, a distal end having a side-facing opening, and a cutting edge extending along a peripheral edge of the opening, the inner member having a proximal end coupled with a drive shaft of a motor disposed in the handpiece, a distal end with a side-facing opening adjacent the opening in the outer member and a cutting edge along a peripheral edge of the opening in the inner member;
positioning the opening in the outer member adjacent a tonsil;
causing electric current to flow to the motor from a battery unit within the handpiece;
continuously rotating the drive shaft to rotate the inner member relative to and within the outer member in response to the flow of electric current to the motor;
cutting anatomical tissue of the tonsil as the cutting edge of the inner member moves past the cutting edge of the outer member as the inner member is rotated within the outer member;
terminating the flow of electric current from the battery unit to the motor;
stopping rotation of the drive shaft to stop rotation of the inner member in response to the termination of electric current flow to the motor;
withdrawing the blade from the patient's mouth; and
disposing of the instrument upon completion of the surgical procedure on the patient.

56. The method of performing a surgical procedure recited in claim 55 wherein said steps of causing and terminating include applying finger pressure to a control unit along an external surface of the handpiece.

57. The method of performing a surgical procedure recited in claim 55 wherein said step of continuously rotating includes starting continuous rotation of the drive shaft instantaneously in response to said step of causing and said step of stopping includes stopping rotation of the drive shaft instantaneously in response to said step of terminating.

58. The method of performing a surgical procedure recited in claim 55 and further including, subsequent to said step of withdrawing, the step of automatically disabling the instrument.

59. The method of performing a surgical procedure recited in claim 55 wherein said step of continuously rotating includes continuously rotating the drive shaft in one direction.

60. The method of performing a surgical procedure recited in claim 55 wherein said step of continuously rotating includes continuously oscillatorily rotating the drive shaft in forward and reverse directions.

61. The method of performing a surgical procedure recited in claim 55 and further including, subsequent to said step of stopping and prior to said step of withdrawing, the steps of contacting the distal end of the outer member with anatomical tissue in the area where tissue of the tonsil was cut and supplying electric current to the distal end of the outer member to cauterize the anatomical tissue in contact with the distal end of the outer member.
